(19) United States
(12) Patent Application Publication
(10) Pub. No.: US 2013/0231647 A1
(43) Pub. Date: Sep. 5, 2013

(54) LASER THERAPY FOR ENDOGENOUSLY ENHANCING VENTRICULAR FUNCTION

(71) Applicant: BFM GROUP INTERNATIONAL GMBH, Lucerne (CH)

(72) Inventors: Hans Michael KLEIN, Dusseldorf (DE); Michael HEKE, New York, NY (US)

(73) Assignee: BFM Group International GMBH, Lucerne (CH)

(21) Appl. No.: 13/785,023

(22) Filed: Mar. 5, 2013

Related U.S. Application Data
(60) Provisional application No. 61/606,560, filed on Mar. 5, 2012.

(51) Int. Cl. A61B 18/20

(52) U.S. Cl. CPC A61B 18/20
USPC 606/14

(57) ABSTRACT

Methods are described for treating a heart using laser therapy by applying low energy pulses to left ventricular. The laser therapy made be used alone or in combination with another therapy, such as cell transplantation or gene therapy, or one or more therapeutic agents, such as a cytokines or growth factors.

### CROSS REFERENCE TO RELATED APPLICATIONS

This application claims priority to U.S. Provisional Patent Application 61/606,560, filed Mar. 5, 2012, which is incorporated by reference herein in its entirety.

### FIELD OF THE INVENTION

This invention relates to methods for treating a heart in a subject in need thereof with laser therapy, either alone or in combination with another therapy or therapeutic agents.

### BACKGROUND OF THE INVENTION

Cardiovascular disease is a leading cause of morbidity and mortality in developing nations. For example, according to the Centers for Disease Control (CDC), in 2008 in the United States, over 616,000 people died of heart disease accounting for almost 25% of deaths. Coronary heart disease is the most common type of heart disease. In 2008, 405,309 people died from coronary heart disease alone in the United States. Furthermore, in 2010, coronary heart disease alone was projected to cost the United States $108.9 billion, including the costs of health care services, medications, and lost productivity. Globally, according to the World Health Organization, an estimated 17.3 million people died from cardiovascular diseases in 2008, representing 30% of all global deaths. Of these deaths, an estimated 7.3 million were due to coronary heart disease. By 2030, the World Health Organization predicts that almost 23.6 million people will die from cardiovascular diseases, mainly from heart disease and stroke, which are projected to remain the single leading causes of death.

Human myocardium cannot compensate for a significant loss of cardiomyocytes. The remodeling process further adds to impairment of cardiac function. Cell therapy, and cell transplantation in particular, has the potential to improve myocardial function and thus, presents a promising means of treating cardiovascular disease.

Transmyocardial revascularization (TMR) is a surgical treatment for cardiovascular disease that has shown limited success. The TMR procedure uses a laser beam to bore holes or channels of approximately 1 mm diameter through the myocardium, typically into the interior left ventricle. These holes or channels extend through the entire heart wall thickness from the outside through to the ventricle. TMR is currently approved by the US Food and Drug Administration for patients with disabling angina for which blockages are too diffuse to be treated with a bypass graft or angioplasty alone and for patients with microvascular disease.

Thus, there is still a need for methods for treating subjects to improve myocardial function.

### SUMMARY OF THE INVENTION

This invention relates to laser therapy, either alone or in combination with another therapy (e.g., cell therapy, such as cell transplantation, or gene therapy) or with one or more therapeutic agents (e.g., cytokines or growth factors) and the therapeutic value of the application of low energy pulses, alone or in combination with another therapy or therapeutic agents, to left ventricular myocardial tissue to treat heart disease.

In one aspect, provided herein are methods of treating a beating heart of a subject in need thereof, the methods include: (a) accessing the beating heart; (b) producing low energy pulses by an energy pulse system to automatically provide energy to strike myocardial tissue only between the R and the T waves of the beating heart; and (c) directing said low energy pulses to strike desired locations on left ventricular tissue of the beating heart. In some embodiments, the methods include prior to step (a) the step of imaging the heart to select the desired locations. In some embodiments, the methods further include performing coronary artery bypass grafting on the subject.

In another aspect, provided herein are methods of treating a heart on heart pump surgery of a subject in need thereof, the methods include: (a) producing low energy pulses by an energy pulse system to provide energy to strike myocardial tissue of the heart; and (b) directing said low energy pulses to strike desired locations on left ventricular tissue of the heart. In some embodiments, the methods include, prior to step (a), the step of imaging the heart to select the desired locations. In some embodiments, the methods further include performing coronary artery bypass grafting on the subject.

In a further aspect, provided herein are methods for preparing a beating heart for transplanting cells and/or administering gene therapy and/or administering one or more therapeutic agents to the beating heart of a subject in need thereof, the methods include: (a) accessing the beating heart; (b) producing low energy pulses by an energy pulse system to automatically provide energy to strike myocardial tissue only between the R and the T waves of the beating heart; and (c) directing said low energy pulses to strike desired locations on left ventricular tissue of the beating heart. In some embodiments, the methods include, prior to step (a), the step of imaging the heart to select the desired locations. In some embodiments, the methods further include performing coronary artery bypass grafting on the subject.

In yet a further aspect, provided herein are methods for preparing a heart on heart pump surgery for transplanting cells and/or administering gene therapy and/or administering one or more therapeutic agents to the heart of a subject in need thereof, the methods include: (a) producing low energy pulses by an energy pulse system to provide energy to strike myocardial tissue of the heart; and (b) directing said low energy pulses to strike desired locations on left ventricular tissue of the heart. In some embodiments, the methods include, prior to step (a), the step of imaging the heart to select the desired locations. In some embodiments, the methods further include performing coronary artery bypass grafting on the subject.

In yet another aspect, provided herein are methods of treating a beating heart, the methods include: preparing or treating the beating heart according to the foregoing methods and transplanting cells to the heart. In some embodiments, the methods further include performing coronary artery bypass grafting on the subject.

In yet another aspect, provided herein are methods of treating a heart on heart pump surgery, the methods include: preparing or treating the beating heart according to the foregoing methods and transplanting cells to the heart. In some embodiments, the methods further include performing coronary artery bypass grafting on the subject.

In yet another aspect, provided herein are methods of treating a beating heart, the methods include: preparing or treating the beating heart according to the foregoing methods and performing gene therapy on the heart. In some embodiments, the methods further include performing coronary artery bypass grafting on the subject.

In yet another aspect, provided herein are methods of treating a heart on heart pump surgery, the methods include: preparing or treating the beating heart according to the foregoing methods and performing gene therapy administering one or more therapeutic agents to the heart. In some embodiments, the methods further include performing coronary artery bypass grafting on the subject.

In yet another aspect, provided herein are methods of treating a beating heart, the methods include: preparing or treating the beating heart according to the foregoing methods and administering one or more therapeutic agents to the heart. In some embodiments, the methods further include performing coronary artery bypass grafting on the subject.

In yet another aspect, provided herein are methods of treating a heart on heart pump surgery, the methods include: preparing or treating the beating heart according to the foregoing methods and administering one or more therapeutic agents to the heart. In some embodiments, the methods further include performing coronary artery bypass grafting on the subject.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description. It is also contemplated that whenever appropriate, any embodiment of the present invention can be combined with one or more other embodiments of the present invention, even though the embodiments are described under different aspects of the present invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram of methods according to certain embodiments of the present invention;

FIG. 2 is a block diagram of the preparation of CD133⁺ bone marrow-derived cells harvested intra-operatively in embodiments of FIG. 1, on the same time scale shown in FIG. 1;

FIG. 3 depicts a schematic view of a left ventricular wall being treated according to certain embodiments of the present invention;

FIG. 4 depicts a schematic view of an area (shown as a box) of left ventricular tissue after being treated according to certain embodiments of the present invention, where Xs represent locations struck by low energy pulses and filled circles represent injection sites;

FIG. 5 are graphs of fluorescence-activated cell sorting (FACS) analyses showing the purity (5A) and viability (5B) of typical bone marrow isolates prepared as in Example 1; and

FIG. 6 illustrates the ECG signal, marker pulse, trigger pulse and firing pulse waveforms for the energy pulse system according to embodiments of the invention.

FIG. 7: Absolute post-procedure increase of left ventricular ejection fraction (LVEF) with (group Acc) and without (group Bcc) cell therapy. After 12 months, the increase of LVEF was significantly larger in group Acc (16.0±3.1% versus 3.7±3.0% in group Bcc, p=0.011). Separated value of group Bcc, outlier included in the statistical group comparison.

FIG. 8: Transmural delayed enhancement (TDE) as a predictor for the postoperative increase of LVEF. TDE in more than three myocardial segments (at screening MRI) resulted in significantly less increase of LVEF after 6 months, as compared to patients with three or less affected segments (1.0±1.4 versus 10.0±4.3, p=0.042).

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

This invention relates to laser therapy, either alone or in combination with another therapy (e.g., cell therapy, such as cell transplantation, or gene therapy) or with one or more therapeutic agents (e.g., cytokines or growth factors) and the therapeutic value of the application of low energy pulses, alone or in combination with another therapy or therapeutic agents, to left ventricular myocardial tissue to treat heart disease.

In one aspect, provided herein are methods of treating a beating heart of a subject in need thereof, the methods include: (a) accessing the beating heart; (b) producing low energy pulses by an energy pulse system to automatically provide energy to strike myocardial tissue only between the R and the T waves of the beating heart; and (c) directing said low energy pulses to strike desired locations on left ventricular tissue of the beating heart. In some embodiments, the methods include prior to step (a) the step of imaging the heart to select the desired locations. In some embodiments, the methods further include performing coronary artery bypass grafting on the subject.

In another aspect, provided herein are methods of treating a heart on heart pump surgery of a subject in need thereof, the methods include: (a) producing low energy pulses by an energy pulse system to provide energy to strike myocardial tissue of the heart; and (b) directing said low energy pulses to strike desired locations on left ventricular tissue of the heart. In some embodiments, the methods include, prior to step (a), the step of imaging the heart to select the desired locations. In some embodiments, the methods further include performing coronary artery bypass grafting on the subject.

In a further aspect, provided herein are methods for preparing a beating heart for transplanting cells and/or administering gene therapy and/or administering one or more therapeutic agents to the beating heart of a subject in need thereof, the methods include: (a) accessing the beating heart; (b) producing low energy pulses by an energy pulse system to automatically provide energy to strike myocardial tissue only between the R and the T waves of the beating heart; and (c) directing said low energy pulses to strike desired locations on left ventricular tissue of the beating heart. In some embodiments, the methods include, prior to step (a), the step of imaging the heart to select the desired locations. In some embodiments, the methods further include performing coronary artery bypass grafting on the subject.

In yet a further aspect, provided herein are methods for preparing a heart on heart pump surgery for transplanting cells and/or administering gene therapy and/or administering one or more therapeutic agents to the heart of a subject in need thereof, the methods include: (a) producing low energy pulses by an energy pulse system to provide energy to strike myocardial tissue of the heart; and (b) directing said low energy pulses to strike desired locations on left ventricular tissue of the heart. In some embodiments, the methods include, prior to step (a), the step of imaging the heart to select the desired locations. In some embodiments, the methods further include performing coronary artery bypass grafting on the subject.

In yet another aspect, provided herein are methods of treating a heart, the methods include: preparing or treating the beating heart according to the foregoing methods and transplanting cells to the heart. In some embodiments, the methods further include performing coronary artery bypass grafting on the subject.

In yet another aspect, provided herein are methods of treating a heart on heart pump surgery, the methods include: preparing or treating the beating heart according to the foregoing methods and transplanting cells to the heart. In some embodiments, the methods further include performing coronary artery bypass grafting on the subject.

In yet another aspect, provided herein are methods of treating a heart, the methods include: preparing or treating the beating heart according to the foregoing methods and performing gene therapy on the heart. In some embodiments, the methods further include performing coronary artery bypass grafting on the subject.

In yet another aspect, provided herein are methods of treating a heart on heart pump surgery, the methods include: preparing or treating the beating heart according to the foregoing methods and performing gene therapy administering one or more therapeutic agents to the heart. In some embodiments, the methods further include performing coronary artery bypass grafting on the subject.

In yet another aspect, provided herein are methods of treating a heart, the methods include: preparing or treating the beating heart according to the foregoing methods and administering one or more therapeutic agents to the heart. In some embodiments, the methods further include performing coronary artery bypass grafting on the subject.

In yet another aspect, provided herein are methods of treating a heart on heart pump surgery, the methods include: preparing or treating the beating heart according to the foregoing methods and administering one or more therapeutic agents to the heart. In some embodiments, the methods further include performing coronary artery bypass grafting on the subject.

The left ventricular tissue of the beating heart may be accessed either epicardially or endocardially. For example, the heart may be accessed thoracoscopically. Alternatively, the beating heart is accessed by performing a left partial thoracotomy, a full sternotomy or a partial sternotomy. The beating heart may be accessed endocardially using a minimally invasive procedure. For example, a catheter is used to direct a fiber to deliver energy pulses in the left ventricle of subject's heart. The catheter can be directed to the heart through various access points in the subject's blood vessels (e.g. transfemorally, transradially or transapically).

The energy pulse system for producing the low energy pulses can be a laser. In some embodiments, the laser is a CO₂ laser (e.g., the pulses have a wavelength of 10.6 μm). Alternatively, the laser is a Ho:YAG laser or an excimer laser. In some embodiments, the energy pulse system that automatically provides energy to strike myocardial tissue only between the R and the T waves of the beating heart is responsive to a controller that is responsive to a sensor comprising an electrocardiogram (ECG) that detects electrical signals from the beating heart. Means, methods and circuitry to synchronize the production of the low energy pulses by a laser to the ECG signal are known in the art, see, e.g., U.S. Pat. No. 6,595,987, which is hereby incorporated by reference in its entirety. Briefly, the laser beam is optically connected with a suitable handpiece or instrument for providing the low energy pulses to the heart. There is some means for sensing the electrocardiogram signal of the beating heart to be synchronized with the laser. This may be a standard ECG device known in the art. The system uses some means for generating a trigger pulse in response to the ECG signal. Typically the trigger pulse is a function of the R wave of the heartbeat cycle generated by the conventional ECG equipment. The heartbeat cycle has four distinct waveforms, the Q, the R, the S, and the T. There are means to for setting the beginning of the trigger pulse so that it occurs in the proper time relationship to the R wave and ends before the T wave to avoid interference with the electrical characteristics of the beating heart. The pulse positioning circuit locates the leading edge of the trigger pulse and a pulse width circuit determines the width so that it extends over only the necessary and safe duration of the heartbeat cycle. The trigger pulse is passed to a laser firing circuit, which then operates the laser to produce the low energy pulses which the surgeon aims precisely to strike at the beating heart preferably during the time between the R and T waves of the heartbeat cycle where the heart is most static, and the accuracy is most assured.

The trigger generator may include a marker pulse circuit for detecting a specific time in the heartbeat cycle of the ECG signal and providing a marker pulse representative of that time. The time may be when the R wave crosses a particular threshold or some time related to that time. The marker pulse circuit may be built in as a part of the readily obtainable ECG unit. The trigger pulse circuit, also is the means for generating the trigger pulse, responds to the marker pulse circuit to provide a trigger pulse whose position in the heartbeat cycle is a function of that specific time in the cycle represented by the marker pulse. The trigger pulse circuit typically includes means for delaying the marker pulse to locate it at a selected position relative to its initial position in the heartbeat cycle, and also contains means for adjusting the delay of the marker pulse to a selected time to create the trigger pulse of the selected position and width. The position of the trigger pulse and its width may be adjusted by a pulse positioning circuit and a pulse width circuit. The laser firing circuit includes a gate which inhibits delivery of the trigger pulse to the laser unless a switch or gate is enabled by the surgeon when he is ready to strike the myocardial tissue of the heart. There is also an arming circuit which further inhibits delivery of the trigger pulse to the laser, even if the surgeon activates the switch or gate unless that arming switch has been actuated. If the arming switch is actuated and the switch or gate is enabled, the next trigger pulse will be directed to fire the laser and provide a low energy pulse to strike myocardial tissue of the heart.

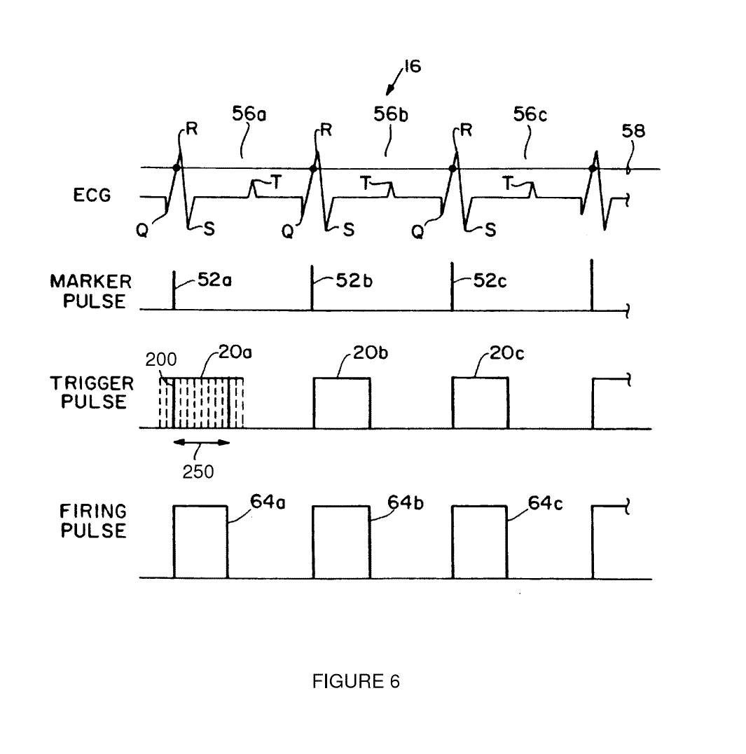

This can be better understood with reference to FIG. 6, where ECG signal 16 may be seen as consisting of a series of heartbeat cycles 56a, 56b, 56c each of which contains the waveforms Q, R, S and T. Where waveform R crosses preselected threshold 58, marker pulses 52a, 52b, 52c are created. Trigger pulses 20a, 20b, 20c are then created by trigger pulse circuit (not shown). The position of the leading edge 200 and the overall width 250 of each trigger pulse 20a, 20b, 20c is determined, respectively, by pulse positioning circuit (not shown) and pulse width circuit (not shown). In response to trigger pulses 20a, 20b, 20c, firing pulses 64a, 64b and 64c are created to energize the laser.

The subjects of the methods described herein can be any suitable mammal, including primates—such as monkeys and humans, horses, cows, cats, dogs, rabbits, and rodents—such as rats and mice. Preferably, the mammal is a human.

In some cases, the subjects of the methods described herein suffer from ischemic heart disease. Examples of ischemic heart disease, include but are not limited to, atherosclerosis of the coronary arteries, angina pectoris, acute coronary syndrome and myocardial infarction. In some cases, the subjects of the methods described herein suffer from dilated cardiomyopathy (DCM). Examples of DCM, include but are not limited to, viral or bacterial endocarditis-myocarditis, or that resulting from any form of primary myocardial hypertrophy.

The terms “treatment” and “treat” as used herein refer to any process, action, application, therapy, or the like, wherein a subject, including a human being, is subjected to medical aid with the object of improving the subject's condition, directly or indirectly. The term “treating” may refer to alleviating symptoms, eliminating recurrence, preventing recurrence, improving symptoms, improving prognosis or a combination thereof. “Treating” also embraces the amelioration of an existing condition. The skilled artisan will understand that treatment does not necessarily result in the complete absence or removal of symptoms. “Treatment” also embraces palliative effects: that is, those that reduce the likelihood of a subsequent medical condition (e.g., congestive heart failure). The alleviation of a condition that results in a more serious condition (e.g., congestive heart failure) is encompassed by this term.

In some embodiments, each low energy pulse provides between less than 1 to 18 joules of energy to strike myocardial tissue of the heart. In some embodiments, each low energy pulse provides between 6 to 16 joules, between 6 to 12 joules, between 8 to 18 joules, between 8 to 16 joules, between 8 to 12 joules, between 10 to 18 joules, between 10 to 16 joules of energy, provides between 10 to 12 joules of energy. In some embodiments, each low energy pulse provides between 1 to 18 joules, between 1 to 16 joules, between 1 to 14 joules, between 1 to 12 joules, between 1 to 10 joules, between 1 to 8 joules, between 1 to 6 joules of energy, between 1 to 4 joules and between 1 to 2 joules of energy. In some embodiments, each low energy pulse provides between 2 to 18 joules, between 2 to 16 joules, between 2 to 14 joules, between 2 to 12 joules, between 2 to 10 joules, between 2 to 8 joules, between 2 to 6 joules of energy and between 2 to 4 joules of energy. In some embodiments, each low energy pulse provides between 4 to 18 joules, between 4 to 16 joules, between 4 to 14 joules, between 4 to 12 joules, between 4 to 10 joules, between 4 to 8 joules and between 4 to 6 joules of energy. In some embodiments, each low energy pulse provides between 6 to 18 joules, between 6 to 16 joules, between 6 to 14 joules, between 6 to 12 joules, between 6 to 10 joules and between 6 to 8 joules of energy. For example, each low energy pulse provides an energy of 1, 2, 3, 4, 5, 6, 7, 8, 9, 10, 11, 12, 13, 14, 15, 16, 17 or 18 joules or any value in between or any range thereof. It will be appreciated that the selection of energy for the low energy pulses in a particular instance will be within the discretion of a person skilled in the art and will depend on many variables such as, without limitation; the particular anatomy of the subject's heart, pre-existing conditions, the severity of symptoms, location and nature of the affected area and the like.

In some embodiments, the methods include the step of imaging the heart to select the desired locations and the regions of the heart for laser therapy. In some embodiments, the imaging step includes computed tomography (CT) scanning and/or magnetic resonance imaging (MRI). In some embodiments, the imaging step includes CT, spiral CT, Positron emission tomography—computed tomography (PET-CT), MRI, and/or high sensitivity 3D echocardiography.

In some embodiments, the desired locations and the regions for laser therapy include viable myocardium, hibernating myocardium or both viable and hibernating myocardium. In some embodiments, the desired locations and the regions of the heart for laser therapy will not include scar tissue, as the therapeutic value, if any, of applying low energy pulses to scar tissue is likely to be outweighed by deleterious effects. Preferably, the imaging is to be used to identify hibernating myocardium, viable myocardium.

It will be appreciated that the number of pulses to be applied will depend on, among other things, the surface area of the left ventricular tissue in need of treatment. Typically, for epicardial applications, about ¼ to ½ (e.g., ⅓) of the outer left ventricle surface area (i.e., at one or more free left ventricular walls (i.e., the anterior, posterolateral, and posterior walls)) will be in need of treatment. In addition, as discussed below, the number of pulses may also depend on the amount of cells to be transplanted, therapeutic agents to be administered and the like. In some embodiments, the number of low energy pulses will range from 10 to 50. In some embodiments, the number of low energy pulses will range from 10 to 35. In some embodiments, the number of low energy pulses will be 10, 15, 20, 25, 30, 35, 40, 45 or 50 or any number in to between. Typically each desired location to be struck by a low energy pulse will be 0.5 cm and 1 cm away from any other desired location to be struck by a low energy pulse. It will be appreciated that the selection of number and locations for the low energy pulses in a particular instance will be within the discretion of a person skilled in the art and will depend on many variables such as those described above as well as, without limitation; the particular anatomy of the subject's heart, pre-existing conditions, the severity of symptoms, location and nature of the affected area and the like.

In some embodiments of the methods described herein, the methods further comprise the step of one or more therapeutic agents to the heart. Preferably, the therapeutic agent is Granulocyte-macrophage colony-stimulating factor (GM-CSF). Other therapeutic agents include angiogenic agents, growth factors or cytokines, but are not limited to, vascular endothelial growth factor (VEGF), acidic fibroblast growth factor (aFGF), basic fibroblast growth factors (bVGF), nerve growth factor (NGF), monocite chemoattractant protein-1 (MCP-1), Angiopoietin-1 (Ang-1), Insulin growth factor I (IGF-I), Insulin growth factor II (IGF-II) and Angiopoietin-2 (Ang-2), a stromal cell derived factor (SDF), a tumor necrosis factor (TNF) (e.g., TNFα), Interleukin 6 (IL-6), a macrophage inflammatory protein (MIP) (e.g., MIP-1α or MIP-1β), a tissue inhibitor of metalloproteinases (TIMP) (e.g., TIMP-1 or TIMP-2), an epidermal growth factor (EGF), a matrix metalloproteinase (MMP) (e.g., MMP-1 or MMP-9), an interleukin (e.g., IL-8), neutrophil-activating protein-2 (NAP-2), or regulated and normal T cell expressed and secreted (RANTES)

In some embodiments of the methods described herein, the methods combine laser therapy and gene therapy to the heart. For example, the gene therapy comprises the step of administering an Adenovirus vector expressing a growth factor, angiogenic factor, a cytokine or the like to the subject. The Adenovirus vector administered expresses, for example, GM-CSF or VEGF.

In some embodiments of the methods described herein, the methods further comprise the step of transplanting cells to the heart. In some embodiments, the cells transplanted are embryonic stem cells, somatic stem cells from cord blood, skeletal myoblasts, hematopoetic stem cells, endothelial progenitor cells, unfractionated bonemarrow-derived mononuclear cells, CD34+ CXCR4⁺ cells, c-kit⁺Lin⁻ cells, Sca-1⁺ cells, C133⁺ progenitor cells, mesenchymal stem cells, resident cardiac stem or progenitor cells, cardiomyocytes derived from HESC or iPSC, adipose tissue derived stem cells (ADSC), epicardial progenitor cells to the cardiomyocyte lineage or Wt1⁺ progenitor cells. In some embodiments, the cells transplanted are bone marrow-derived cells. In some embodiments, the bone marrow-derived cells are autologous bone marrow cells. In some embodiments, autologous bone marrow cells are obtained intra-operatively. In some embodiments, the bone marrow-derived cells include CD133⁺ bone marrow cells. In some embodiments, the cells to be transplanted (e.g., autologous CD133⁺ bone marrow cells) are at least 50% viable. In some embodiments, the cells to be transplanted (e.g., autologous CD133⁺ bone marrow cells) are at least 60% viable. In some embodiments, the cells to be transplanted (e.g., autologous CD133⁺ bone marrow cells) are at least 70% viable. In some embodiments, the cells to be transplanted (e.g., autologous CD133⁺ bone marrow cells) are at least 75% viable. In some embodiments, the cells to be transplanted (e.g., autologous CD133⁺ bone marrow cells) are at least 80% viable. In some embodiments, the cells to be transplanted (e.g., autologous CD133⁺ bone marrow cells) are at least 85% viable. In some embodiments, the cells to be transplanted (e.g., autologous CD133⁺ bone marrow cells) are at least 90% viable. In some embodiments, the cells to be transplanted (e.g., autologous CD133⁺ bone marrow cells) are at least 95% viable. In some embodiments, the cells to be transplanted (e.g., autologous CD133⁺ bone marrow cells) are more than 98% viable. In some embodiments, the cells to be transplanted to (e.g., autologous CD133⁺ bone marrow cells) are at least 75% pure. In some embodiments, the cells to be transplanted (e.g., autologous CD133⁺ bone marrow cells) are at least 80% pure. In some embodiments, the cells to be transplanted (e.g., autologous CD133⁺ bone marrow cells) are at least 85% pure. In some embodiments, the cells to be transplanted (e.g., autologous CD133⁺ bone marrow cells) are at least 90% pure. In some embodiments, the cells to be transplanted (e.g., autologous CD133⁺ bone marrow cells) are at least 95% pure. In some embodiments, the cells to be transplanted (e.g., autologous CD133⁺ bone marrow cells) are more than 98% pure.

In some embodiments, a total of between 0.5×10⁶ cells and 50×10⁶ cells will be transplanted. In some embodiments, a total of between 2×10⁶ cells and 30×10⁶ cells (e.g., a total of 6×10⁶ cells) will be transplanted.

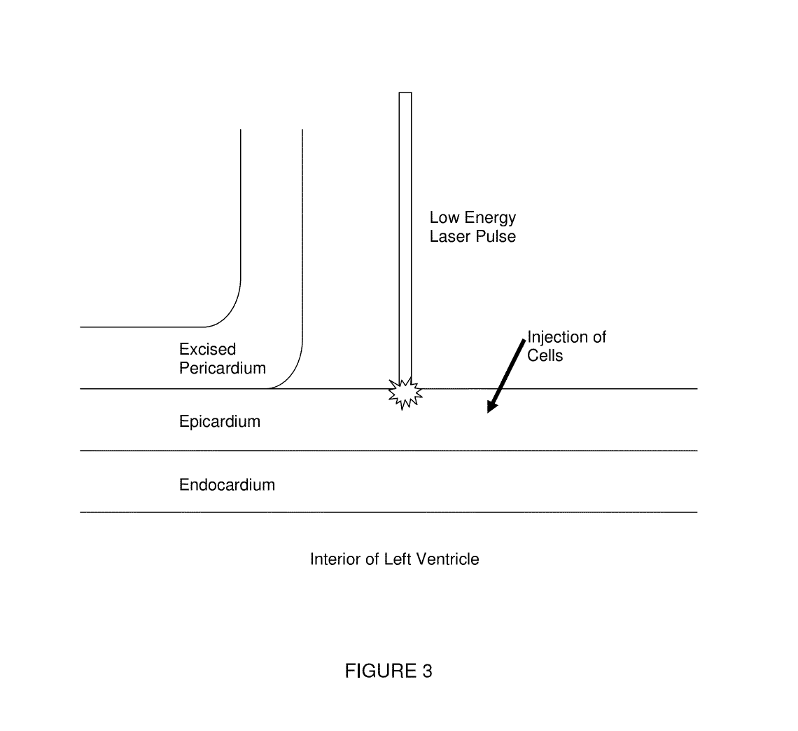

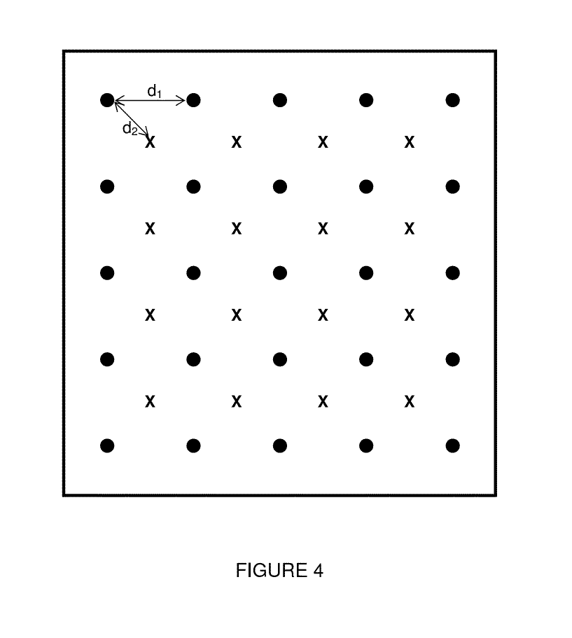

Typically, the step of transplanting cells or administering therapeutic agents to the heart will include injecting the cells or therapeutic agents, respectively, from outside the heart directly into the heart epicardium adjacent to one or more of the locations struck by a low energy pulse (See FIGS. 3 and 4). Alternatively or additionally, the step of transplanting the cells or administering therapeutic agents may be performed by intravenous administration, intracoronary administration, transendocardial administration or transepicardial administration. In general, there will be 10, 15, 20, 25, 30 or 35 injections or any number in between. Typically, each location struck by a low energy pulse will have 1, 2, 3 or 4 injections of transplanted cells or therapeutic agents adjacent to it. Generally, each injection will be within 0.5 to 1 cm of at least one location struck by an energy pulse. It will be appreciated that the quantity, location and number of injections in a particular instance will be within the discretion of a person skilled in the art and will depend on many variables such as, without limitation; the particular anatomy of the subject's heart, pre-existing conditions, the severity of symptoms, location and nature of the affected area and the like.

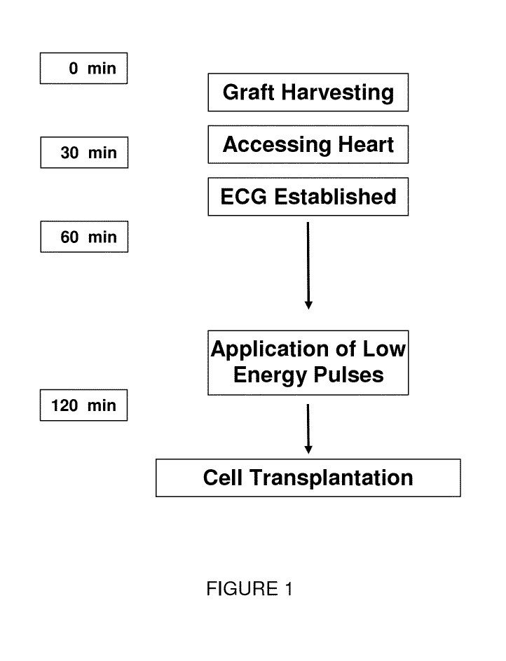

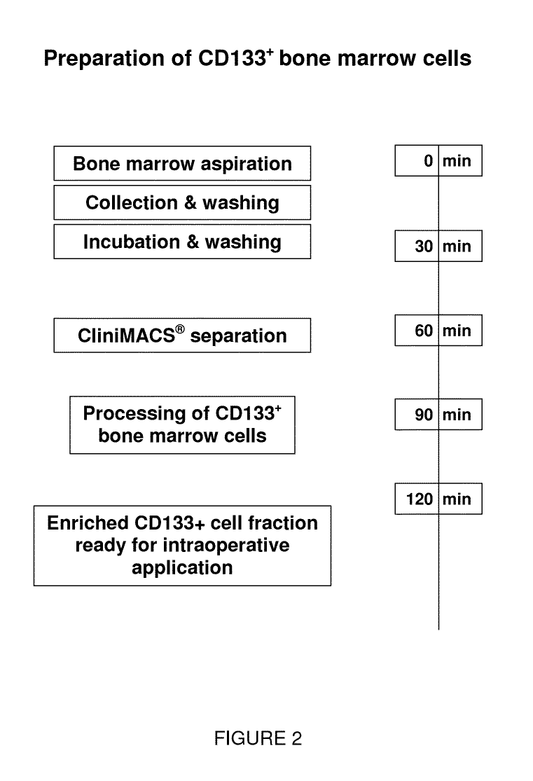

There is shown in FIG. 1 a block diagram of methods according to certain embodiments of the present invention and the time at which each step is performed. Immediately after anesthesia, the bone marrow is harvested and processed as shown in FIG. 2 and described in Example 1. The heart is accessed and an ECG is established to control the energy pulses to strike myocardial tissue only between the R and the T waves of the beating heart, when the beating heart is most static and to avoid interference with the electrical characteristics of the beating heart wave. The myocardial region of interest is pretreated by the application of low energy pulses, and then cells are transplanted to the region of interest.

Referring now to FIG. 3, which is a schematic view of a left ventricular wall being treated according to certain embodiments of the present invention. The pericardium has been excised to allow direct access to the myocardial tissue. Low energy laser pulses are applied to the epicardial surface of the myocardium. Cells or therapeutic agents are then injected into the epicardium of the myocardial tissue. The intramyocardial injection during cardiac surgery by the means of transepicardial application is fast and easily performed. Moreover, after administration this way the cells or therapeutic agents are primarily located within the region of interest—the myocardium. The injection of cells or therapeutic agents directly into the epicardium not only assures them reaching the target region but also ensures that they remain in this area for the time being.

Referring now to FIG. 4, which is a schematic view of an area (shown as a box) of left ventricular tissue after being treated according to certain embodiments of the present invention, where Xs represent locations struck by low energy pulses and filled circles represent injection sites. Furthermore, d₁ represents the distance between adjacent locations struck by low energy pulses or adjacent injections, and will typically be about 0.5 cm to 1 cm. In addition d₂ represents the distance between a location struck by a low energy pulse and an adjacent injection, and will typically be less than about 0.5 cm to less than about 1 cm. Typically, each location struck by a low energy pulse will have 1, 2, 3 or 4 injections adjacent to it. However, it may be the case that a number of locations struck by a low energy pulse will have 0 or greater than 4 injections of transplanted cells adjacent to it. It is also understood that more than one location struck by a low energy pulse may share at least one injection among them. In addition to the factors above, for cell transplantation the number of injections may be influenced by the total volume of cells available for transplantation and the volume for each injection. For example, if there are a total of 5 mL of cells available for transplantation and each injection is 0.2 mL of cells, then up to 25 injections are possible.

All sequence citations, accession numbers, references, patents, patent applications, scientific publications or other documents cited are hereby incorporated by reference.

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### EXAMPLES

### Example 1

### Intra-Operative Harvesting and Processing of Autologous CD133+ Bone Marrow Cells for Transplantation

Patient bone marrow was aspirated after the induction of anesthesia (See FIG. 2); heparin coated syringes were used to obtain 200 to 400 mL of bone marrow aspirate from the iliac crest. The aspirate was collected in blood bags and washed with EDTA/phosphate buffered saline/25 mL 20% human albumin The cell suspension was filtered to remove bone specula and then processed to select for CD133⁺ cells by a GMP-certified cell selection unit (CliniMACS® Cell Separation System; Miltenyi Biotec, Bergisch Gladbach Germany) on a laminar flow bench within the operating theatre. After approximately 160 minutes, the enriched mononuclear cells were ready for intramyocardial injection.

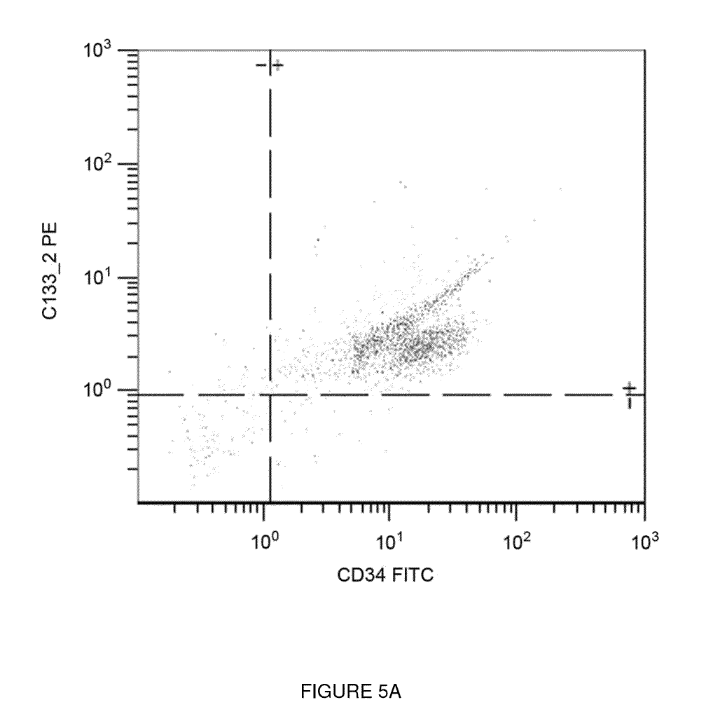

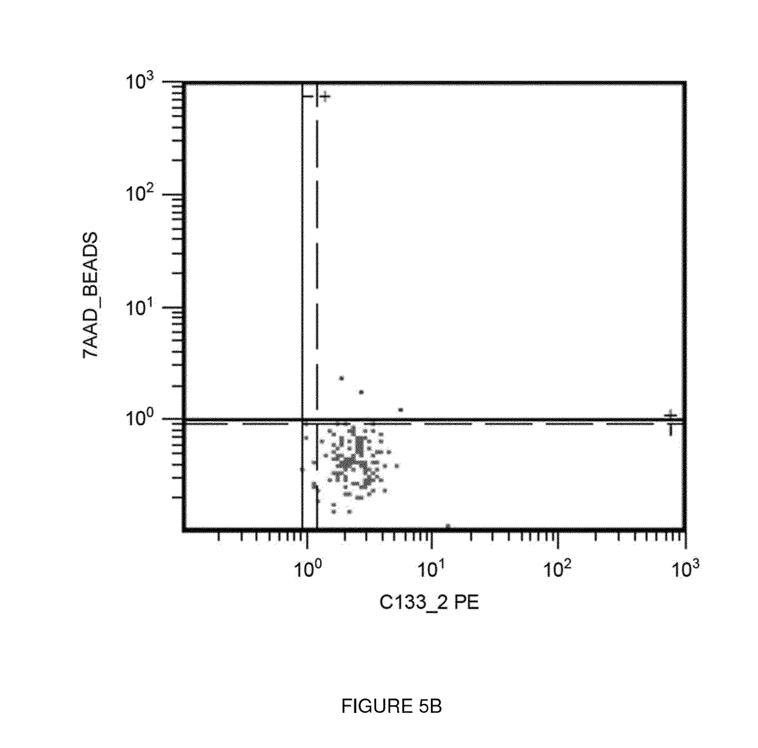

Aliquots from the bone marrow aspirate and the injection cell fraction were collected. The number of mononuclear cells was registered by a cell counter (Sysmex, Mundelein, Ill., USA). Aliquots were analyzed by fluorescence-activated cell sorting (FACS) using anti-CD133, anti-CD34, anti-CD45 and propidium iodide (Miltenyi Biotec). Fluorescence-activated cell sorting (FACS) reveals CD133⁺ cell purities of greater than 98% (FIG. 5A) and cell viabilities of greater than 96% (FIG. 5B).

### Example 2

### Therapeutic Value of Laser Therapy Plus Cell Transplantation

The INSTEM Clinical Trial at the Clinic for Cardiovascular Surgery, University Hospital Düsseldorf (H. M. Klein et al./Multimedia Manual of Cardiothoracic Surgery/doi:10.1510/mmcts.2009.003947), was aimed at examining safety, feasibility and regenerative potential of intraoperative CD133+ cell isolation and laser-supported transplantation in coronary artery bypass grafting (CABG) patients with ischemic cardiomyopathy.

In a prospective multicenter trial, patients with severe ischemic cardiomyopathy (n=39) underwent conventional CABG therapy supported by laser therapy and subsequent CD133+ cell transplantation. The follow-up lasted 12 months. Moreover, a case control study was performed to evaluate the regenerative potential of this cell therapy approach.

The intraoperative cell isolation protocol (see FIG. 2) resulted in a high-quality cell product. During the follow-up, no procedure-related adverse events occurred. Freedom from angina was achieved and quality of life notably improved after therapy (p<0.0001). Furthermore, the left ventricular ejection fraction (LVEF) significantly increased after surgery (p<0.015) and was significantly enhanced in the cell therapy group versus control (p<0.011, after 12 months).

The protocol for intraoperative CD133+ cell isolation and transplantation following laser treatment in CABG patients with ischemic cardiomyopathy proved to be feasible and safe. Moreover, it was demonstrated to beneficially affect the myocardial function.

39 patients with severe ischemic cardiomyopathy, resulting in a decreased left ventricular ejection fraction (LVEF)>15% and <35%, were enrolled in our prospective phase two multicenter trial. The study protocol for all investigational sites was approved by the national competent authority, the Paul Ehrlich institute, as well as by the local ethics committee.

In order to evaluate the results of this therapeutic approach, a case control study was conducted after the first 18 patients had been operated. The control group (group B) consisted of all patients with severe ischemic cardiomyopathy (LVEF >15% and <35%), who underwent isolated CABG during the same time period. Statistical matching led to 14 pairs of study and control group patients (group Acc and group Bcc).

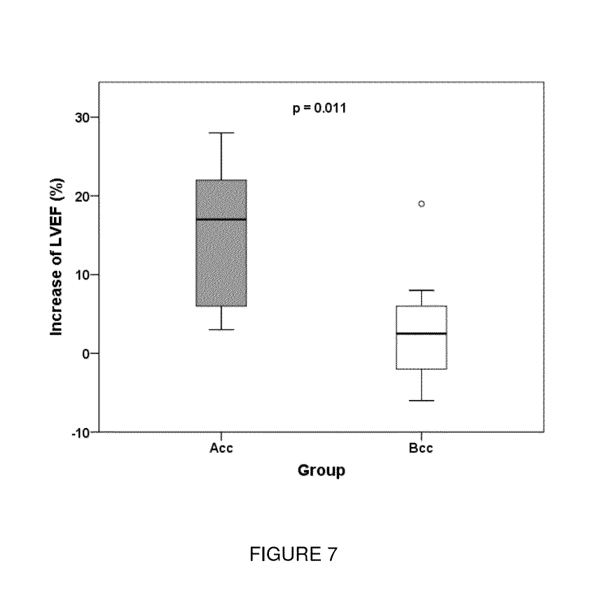

In order to separate the effects of laser therapy and cell therapy from the impact of CABG on the postoperative increase of the systolic cardiac function, the LVEF of group Acc was compared with the LVEF of group Bcc. Therefore, echocardiography was performed in both groups after 12 months and revealed increased LVEF values versus the preoperative data. This finding reached significance only in group Acc (40.7±2.1% versus 25.9±1.3%, p<0.0001), but not in group Bcc (34.2±2.2% versus 31.3±1.5%, p=0.134). Furthermore, the absolute increase of LVEF during the follow-up was compared between the two groups and a significant difference was found (16.0±3.1% in group Acc versus 3.7±3.0% in group Bcc, p=0.011) (FIG. 7).

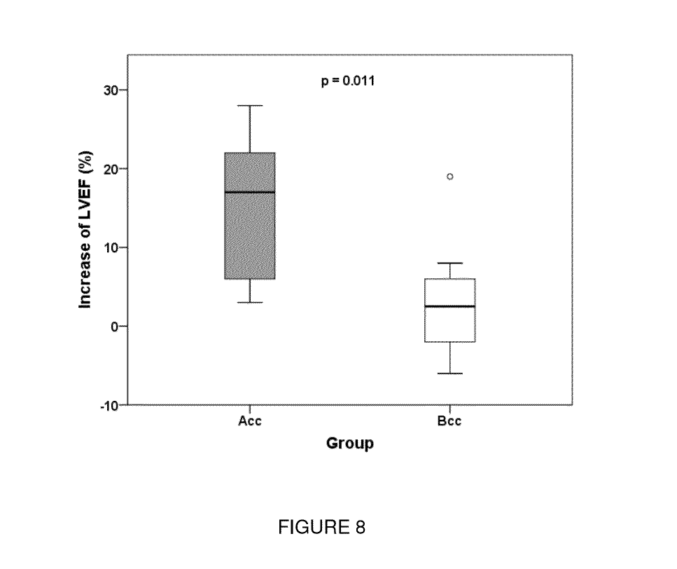

An extended area of transmural delayed enhancement (TDE) in the pre-operative magnetic resonance imaging analyses was shown to be a predictor for less beneficial effects of this therapy (p<0.042). Thus, the degree of improvement may be predicted by preoperative magnetic resonance imaging assessing the total area of hibernating myocardium (FIG. 8).

### Example 3

### Intra-operative Harvesting and Processing of Autologous CD133+Bone Marrow Cells for Transplantation

This example expands on the results of the INSTEM clinical trial discussed in the previous Example and further demonstrates how laser therapy amplifies existing endogenous repair mechanisms. In this approach laser therapy is combined with the administration of Granulocyte-macrophage colony-stimulating factor (GM-CSF) rather than transplanting CD133+ cells as in the INSTEM trial. The results of the GM-CSF-triggered, endogenous cell therapy are compared to that of transplanted CD133+ cells in the INSTEM trial. In addition, the benefits rendered by laser therapy alone, versus complementing GM-CSF pre-treatment, versus control are compared.

Epicardial laser treatment in a surgical approach is compared in trials to endocardial laser treatment in a non-invasive, endoscopic approach.

### Animal Plan:

### Day 0: Induction of Myocardial Infarction in the LV Anterior Wall

Myocardial infarction is induced using a standardized “closed chest occlusion-reperfusion protocol” according to standard protocols. Briefly, after selective coronary angiography, a balloon is inflated in the mid part (after the origin of the first major diagonal branch) of the left anterior descending coronary artery (LAD) for 90 minutes followed by reperfusion.

### Day 3: Baseline MRI

Animals undergo standard cardiac MRI to determine ejection fraction, volume measurements, area at risk, etc.

### Day 30:

### Pre Treatment MRI

Laser Therapy in Combination with GM-CSF or Cell Treatment

Animals undergo laser therapy plus intramyocardial transplantation of GM-CSF or Cell Treatment into the LV anterior wall using a minimally invasive, thoracotomy based approach.

### Post Treatment MRI

### Day 37-40: Final Assessment MRI

Animals undergo follow up cardiac MRI to determine ejection fraction, volume measurements, area at risk, etc.

### Day 40: Animal Harvest and Post Mortem Assessment

Animals are sacrificed and hearts are harvested for post mortem assessment. Histology, Immunohistochemistry, PCR, etc are performed to assess the impact on myocardial infarction with specific regards to tissue regeneration, remodeling, scar size, etc.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treating a beating heart of a subject in need thereof, comprising:
accessing said beating heart;
producing low energy pulses by an energy pulse system to automatically provide energy to strike myocardial tissue only between the R and the T waves of the beating heart;
directing said low energy pulses to strike desired locations on left ventricular tissue of said beating heart.

2. The method of claim 1, wherein the subject in need thereof suffers from ischemic heart disease or dilated cardiomyopathy.

3. The method of claim 1, wherein the heart is accessed epicardially.

4. The method of claim 1, wherein the heart is accessed endocardially.

5. The method of claim 1, wherein said low energy pulses are produced by a CO₂ laser and have a wavelength of 10.6 μm.

6. The method of claim 1, wherein said low energy pulses are pulses having an energy of 6 to 18 joules.

7. The method of claim 1, wherein said low energy pulses are pulses having an energy of 1 to 8 joules.

8. The method of claim 1, wherein 10 to 50 low energy pulses are directed to strike left ventricular tissue.

9. The method of claim 1, said desired locations of said beating heart comprises hibernating myocardium.

10. The method of claim 1, wherein said subject is human.

11. The method of claim 1, wherein each desired location is between 0.5 and 1 cm away from any other desired location.

12. The method of claim 1, further comprising imaging said heart to select said desired locations, prior to the step of accessing said heart.

13. The method of claim 12, wherein said imaging step comprises computed tomography (CT) scanning or magnetic resonance imaging (MRI).

14. The method of claim 1, further comprising the step of performing coronary artery bypass grafting on said subject.

15. The method of claim 1, further comprising the step of transplanting cells to said beating heart.

16. The method of claim 15, wherein said cells comprise autologous bone marrow-derived cells.

17. The method of claim 16, wherein said bone marrow-derived cells comprise CD133⁺ bone marrow cells.

18. The method of claim 15, wherein said step of transplanting comprises injecting cells into left ventricular tissue adjacent to said desired locations.

19. The method of claim 18, wherein each desired location has between 1 and 4 injections adjacent to it.

20. The method of claim 18, wherein each injection is less than 0.5 to less than 1 cm of at least one desired location.

21. The method of claim 1, further comprising the step of administering a therapeutic agent to said beating heart.

22. The method of claim 21, wherein said therapeutic agent comprises a cytokine.

23. The method of claim 21, wherein said therapeutic agent comprises a growth factor.

24. The method of claim 21, wherein said therapeutic agent comprises Granulocyte-macrophage colony-stimulating factor (GM-CSF).

25. The method of claim 21, wherein said therapeutic agent comprises vascular endothelial growth factor (VEGF).

26. The method of claim 21, wherein said step of administering comprises injecting the therapeutic agent into left ventricular tissue adjacent to said desired locations.

27. The method of claim 26, wherein each desired location has between 1 and 4 injections adjacent to it.

28. The method of claim 26, wherein each injection is less than 0.5 to less than 1 cm of at least one desired location.

29. A method of treating a heart on heart pump surgery of a subject in need thereof, comprising:
producing low energy pulses by an energy pulse system to provide energy to strike myocardial tissue of the heart;
directing said low energy pulses to strike desired locations at one or more free left ventricular walls of said heart.
